(19) United States
(12) Patent Application Publication
(10) Pub. No.: US 2005/0216044 A1
(43) Pub. Date: Sep. 29, 2005

(54) Total occlusion recanalization facilitating device

(76) Inventor: Mun K. Hong, New York, NY (US)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 11/090,435

(22) Filed: Mar. 25, 2005

Related U.S. Application Data
(60) Provisional application No. 60/556,343, filed on Mar. 25, 2004.

(51) Int. Cl. A61B 17/22

(52) U.S. Cl. 606/159; 604/528

(57) ABSTRACT

A system for recanalizing chronic total occlusions comprises a catheter having multiple channels that are substantially coextensive distally and distensible. In addition, the catheter may have a bullet-shaped distal tip and/or torquing grooves in the proximal and distal shaft.

### CROSS-REFERENCE TO RELATED APPLICATION

This application corresponds to U.S. provisional patent application Ser. No. 60/556,343, filed Mar. 25, 2004, the entirety of which is incorporated herein by reference.

### FIELD OF THE INVENTION

This application is directed to a recanalization angioplasty system for total chronic occlusions. More particularly, this application is directed to a system for guiding and facilitating the recanalization of chronic total occlusions, wherein a multi-lumen, multi-functional angioplasty device is used to recanalize chronic total occlusions.

### BACKGROUND OF THE INVENTION

Chronic total occlusions, in the coronary and peripheral arteries, as well as in other vascular structures, such as the veins or dialysis fistulae, represent a challenge for percutaneous treatment, a preferred revascularization option compared with bypass surgery. With continuing improvements in equipment, mainly stiffer and hydrophilic guidewires specifically developed for chronic total occlusions, and in operator skills and techniques such as subintimal dissection, success rates are increasing. Although the success rate has improved, the procedure still requires many manipulations of multiple equipments depending on the complexity of the occlusion. However, there are still a sizeable number of patients, in whom the percutaneous approach is unsuccessful, mainly due either to the inability of the guidewire to completely cross the chronic total occlusions or to the inability of subsequent angioplasty equipment to traverse the fibrocalcific total occlusions over the successfully negotiated guidewires. Those patients without a successful percutaneous recanalization need to undergo bypass surgery or experience continuing symptoms from the occlusions. Furthermore, patients with failed attempts at recanalization may have increased later mortality. Despite the availability of new devices to attempt the recanalization of chronic total occlusions without a guidewire or to combine ablative energy with a guidance system, these devices may increase the complication rates of the procedure. In addition, there are instances when the intravascular location of the distal tip of the guidewire is in question and angioplasty of the pathway over the guidewire may result in perforation of the vessel. Thus, there is a need for a new device to further increase the success rate without added risk to recanalize chronic total occlusions.

What is difficult about chronic total occlusion angioplasty is that there may be a distortion of the regular vascular architecture and that there may be multiple small non-functional channels throughout the occlusions rather than one central lumen for recanalization. Thus, the conventional approach of looking for the single channel in the center of the occlusion may account for many of the failures. Furthermore, these spontaneously recanalized channels may be responsible for failures due to their dead-end pathways and misdirecting of the guidewires. Once a “false” tract is created by a guidewire, subsequent attempts with different guidewires may continue to follow the same incorrect path, and it is very difficult to steer subsequent guidewires away from the false tract.

These obstacles account for majority of the failures with the conventional approach for chronic total occlusions, which consists of a chosen guidewire and a single-lumen catheter or balloon over the guidewire for support. This approach allows mainly one shot with the guidewire and single-lumen catheter, as unsuccessful passage of the guidewire would result in creation of a false tract and inability to easily manipulate the system into a different, correct path. This conventional approach is further hampered by the need to either remove the initially chosen guidewire with unsuccessful pathway or remove the single-lumen support catheter to be used with another guidewire if the operator chose to leave the initial guidewire in the “false” track or tract. This exchange maneuver is necessary, as even the new guidewires designed for chronic total occlusions still benefit from an encasing support catheter or balloon. The absence of multi-lumen co-axial support catheter demands this lengthy, cumbersome approach. In the era of reliable stents, especially drug-eluting stents, and in particular, in chronic total occlusions of the peripheral arteries, even subintimal dissection can be successfully treated with creation of a new lumen. Thus, it is desirable to have a novel device that would enable crossing of the guidewires throughout the length of the occlusion, as long as the distal tip of the guidewire is intravascular.

Another equally important failure mode even after a guidewire successfully crosses a chronic total occlusion is the inability to advance a balloon or another angioplasty equipment over the guidewire due to the fibrocalcific composition of the chronic total occlusion, mainly both at the “entry” point and at the “exit” segment of the chronic total occlusion. Even with balloon inflations throughout the occlusion, many times there is no antegrade flow of contrast injected, possibly due to the recoil or insufficient channel creation throughout the occlusion. The worst case scenario is that the balloon inflations occurred outside the vessel. Although an ablative energy source, such as a laser, may be useful, it is not universally available and its use may result in perforation of the blood vessel. Thus, a support catheter with inherent ability to successfully traverse the fibrocalcific components at either end of a chronic total occlusion over the guidewire would greatly facilitate the subsequent successful stent implantation for a definitive revascularization.

Finally, it is imperative that the revascularization attempt does not result in a devastating complication, such as perforation of the vessel. This important issue demands a means afforded by the device to confirm the intravascular location of the distal guidewire prior to the use of balloon inflations, ablative energy, or atherectomy devices.

For all these reasons, it is desirable to have a novel catheter that would allow multiple, non-overlapping attempts at successfully crossing a total occlusion and further enable subsequent advancement of the device reliably across the occlusion. In instances where the intravascular location of the guidewire is in question, the device should enable a method to confirm the intravascular location.

### OBJECTS OF THE INVENTION

It is an object of the invention to provide a recanalization facilitation device for chronic total occlusions.

It is also an object of the invention to provide a recanalization system for chronic total occlusions wherein a multi-lumen, multi-functional device will provide multiple, substantially parallel channels for different guidewire pathways or tracts without the possibility of following false tracts created by previous guidewire attempts.

It is a further object of the invention to provide a recanalization system for chronic total occlusions wherein a multi-lumen, multi-functional device will provide both the “dottering” and a resultant substantial channel creation in the chronic total occlusions for subsequent definitive recanalization.

It is a yet further object of the invention to provide a recanalization system for chronic total occlusions wherein the distal tip of the catheter is tapered and the shaft at proximal and distal segments has circumferential grooves for manual torquing of the device for advancement through the chronic total occlusion.

It is a yet further object of the invention to have distensible lumens that could be stretched with a low-profile balloon catheter or other catheter over the guidewire so that it could facilitate complete crossing of the chronic total occlusion or to confirm the intravascular location of the guidewire to make the procedure safer.

It is a yet further object of the invention to provide a multi-lumen catheter that aids with multiple guidewire insertions in bifurcation or trifurcation areas or with the insertion of a “buddy” guidewire.

These and other objects of the invention will become more apparent from the discussion below.

### SUMMARY OF THE INVENTION

Current percutaneous treatment of chronic total occlusions is challenging and can be associated with up to 30% failure rate. Even a successful procedure requires much patience and skill on the part of the operator and much resource consumption with many different guidewires, catheters, balloons, and other angioplasty equipment. An ideal device to recanalize chronic total occlusions as well as acute, but complex occlusions should possess all the properties to overcome the obstacles currently associated with this procedure, including the guidance and simultaneous multiple attempts for successful crossing with the guidewires, the ability to reliably cross the occlusion with the device following guidewire crossing, and sufficient channel or pathway created through the occlusion for subsequent stent implantation while ensuring the safety of the procedure by minimizing the risk of vessel perforation.

The invention herein is directed to a recanalization facilitation catheter device for total occlusions, both in the coronary arteries and in peripheral arteries, as well as in other intravascular structures. This catheter with multiple, substantially parallel lumens traversing the entire length of the catheter from the proximal end to the distal tapered end, will enable simultaneous, multiple attempts with one or more different guidewires and, thus, greatly facilitate the successful traversing of a guidewire through the occlusion. In addition, the optional presence of a series of circumferential, skewed grooves on the proximal shaft and similar grooves at the distal shaft immediately proximal to a distal tapered tip would enable “dottering” of the device with manual torquing of the entire catheter through the fibrocalcific chronic total occlusion and will facilitate the passage of a catheter through a chronic total occlusion over a guidewire.

Preferably the lumens are distensible so that when there is difficulty advancing the catheter through an occlusion, possibly due to relatively bulky nature of the device or when the intravascular location of the guidewire is in question, a low-profile balloon catheter or other catheter could be advanced over the guidewire by stretching this lumen. The balloon catheter could be advanced to extend the balloon beyond the distal tip of the catheter to further facilitate complete crossing of the chronic total occlusion or to confirm the intravascular location with contrast injection. In addition, the device of the invention can be used in non-total occlusions, where a series of guidewires would be needed, such as in bifurcation areas or when a second guidewire for support (a “buddy” wire) is desirable.

An important aspect of the current invention is the presence of multiple, substantially parallel, longitudinally extending lumens. These lumens permit simultaneous or consecutive attempts at multiple tracts, where subsequent guidewires are steered away from the previous tracts without the possibility of falling into the false tracts. This feature may also improve crossing of the chronic total occlusions with the device when more than one guidewire either traverses the occlusion completely (at least one successful crossing) or even partially by providing the “anchoring” in the fibrocalcific occlusion.

In another aspect of the invention the distal tip of the recanalization catheter is preferably tapered or “bullet” shaped. The distal tip optionally comprises a physio-logically acceptable rigid or semi-rigid polymeric or metal surface, such as nitinol or stainless steel. A distal tip having such a shape would facilitate advancing the catheter with proximal torquing, optionally with a “screwing” effect and/or dottering, to create a substantial lumen without the recoil associated with balloon inflation.

In another aspect of the invention a series of diagonal, circumferential grooves are positioned immediately proximal to the distal tapered tip and/or from about 0.5 cm to about 2.0 cm distal to the proximal end of the catheter shaft. Such grooves at the distal end should facilitate manual torquing of the catheter and successful crossing of the fibrocalcific total occlusion in combination with the tapered tip. The grooves at the proximal end of the catheter shaft are specifically intended to make it easier for an operator to grasp and hold the catheter shaft and torque the catheter.

Optionally the proximal end of the catheter shaft may have other functional features to assist an operator in grasping the catheter shaft. Such features could include a roughened surface, knobs, nubs, or other projections, ridges, or the like.

In another aspect of the invention the lumens of the catheter can be stretched to accept a low-profile balloon catheter or other catheter which would be inserted after apparently successful crossing of an occlusion with a guidewire. Optionally the proximal end of the catheter of the invention and/or one or more of the longitudinally extending lumens could be flared to facilitate insertion of a low-profile balloon catheter or other catheter over a guidewire. These features would ensure the safety of the procedure by allowing a lower profile device rather than a bulkier recanalization system to cross the entire occlusion and to confirm the intravascular location of the distal guidewire.

Another application of the device of the invention would be in addressing bifurcation or trifurcation areas, where “tangling” of the guidewires would be prevented by the multiple co-axial channels for multiple guidewires in different branches. This application could also facilitate the placement of a “buddy” guidewire next to an existing guidewire in non-total occlusions. For these applications, the channels in the catheter do not necessarily have to be perfectly parallel.

According to the invention the body of the recanalization catheter can be introduced using known techniques over a movable guidewire (or equivalent structure) which has been previously positioned at a desired location within a patient's vascular system. The catheter is manipulated until the distal portion of the catheter lies within the location of interest, for example, adjacent an occlusion or stenotic lesion. Once the catheter is in position, either the guidewire can be advanced into the occlusion or, if a stiffer guidewire is required, the guidewire can be retracted (i.e., pulled in the proximal direction) so that it evacuates the lumen in the distal region but remains within a lumen in the proximal region, wherein a second guidewire is advanced through another lumen.

### BRIEF DESCRIPTION OF THE DRAWINGS

FIG. 1 is a perspective, partly cross-sectional view of one embodiment of the present invention, where there are multiple, substantially parallel lumens traversing the entire length of the device (variable number of channels, but a minimum number of two channels) and a distal tip tapered or shaped like a bullet, with or without a physiologically acceptable metal or other rigid or semi-rigid surface. Immediately proximal to the distal tip and in the proximal segment of the shaft, there are diagonal, circumferential grooves for torquing the entire device for “dottering”;

FIGS. 2 to 5 are each a cross-sectional view along line 2-2 in FIG. 1, showing a variable number of channels, with a minimum number of two channels;

FIG. 6 is a view of the proximal surface of the catheter of FIG. 1, showing the channels closed by one-way valves, with the entrance to each channel perforated by slits;

FIG. 7 is a perspective view of the proximal end of another embodiment of the invention having a flared end so that the channels can be stretched and accommodate an optional low-profile balloon catheter or other catheter;

FIGS. 8 and 9 are each a perspective view of the device creating a substantial lumen over the guidewire in the proximal fibrocalcific segment of a chronic total occlusion. In FIG. 8, due to forward pushing of a catheter of the invention over a guidewire, the distal tip is wedged in a fibrocalcific occlusion. With subsequent torquing of the device and transmission of the torque with simultaneous forward pushing (FIG. 9), the catheter is further advanced into the occlusion;

FIG. 10 represents a cross-sectional view of the catheter with a low-profile balloon catheter inserted over a guidewire in one of the lumens, with enlargement of this lumen and compression of the other lumens.

FIG. 11 shows the low-profile balloon catheter traversing the occlusion, with the balloon extending beyond the occlusion over the guidewire; and

FIG. 12 shows the guidewire removed and the intravascular location confirmed by injection of contrast agent into the central lumen of the low-profile balloon catheter.

### DETAILED DESCRIPTION OF THE INVENTION

According to the invention, a recanalization facilitation system is advanced percutaneously through a guiding catheter over at least one guidewire to a position immediately proximal to a chronic total occlusion. The placement of guiding catheters and/or guidewires is well known to those skilled in the art.

After the recanalization facilitation system is positioned at the proximal segment of the chronic total occlusion, the operator initially manipulates a selected guidewire through one of the lumens into the total occlusion. If the operator is successful in crossing the occlusion, the operator then tries to advance the catheter device over the guidewire. This technique is well known to those skilled in the art.

If the operator is not successful in advancing the catheter device over a guidewire into the occlusion due to the fibrocalcific lesion composition, the operator can then torque the proximal catheter shaft while advancing the device gently so that the catheter distal tip acts as a screw and wedges itself in the fibrocalcific occlusion. With further torquing and forward pushing, the device may cross the occlusion. Even without complete crossing with the catheter device, the initiation of the channel in the chronic total occlusion would enable a low-profile balloon catheter to be advanced over the guidewire for subsequent balloon inflation and stent implantation. In addition, if there is any question about the intravascular location of the distal tip of the guidewire, a low-profile balloon catheter could be inserted over the guidewire and advanced to position the balloon beyond the occlusion. With temporary removal of the guidewire and contrast injection into the central lumen of the low-profile balloon catheter, the intravascular location can be confirmed prior to further manipulation of the device or inflation of the balloon to minimize the risk of the procedure. Finally, if the device still cannot be advanced due to its relative bulk, the low-profile balloon could be inflated beyond the occlusion to provide a distal anchor for the device to be advanced through the occlusion.

On the other hand, if the operator is not successful in crossing the occlusion with the first guidewire through the initially chosen lumen, either the same guidewire or a different guidewire can be inserted into a second lumen and then manipulated to cross the occlusion. Preferably the first guidewire is left in the first lumen and a second guidewire is then used to further prevent the second guidewire following the path of the first guidewire. Also, the first wedged guidewire may be used as an anchor to increase the likelihood of the device to be advanced through the occlusion. This process can be repeated with one or more additional guidewires at this point. When a guidewire finally crosses the occlusion, then the procedures above with the catheter and/or a low-profile balloon catheter can be followed.

The catheter shaft will comprise one or more flexible tubular members having axial lumens formed therein, as described in more detail below. A catheter body will be suitable for insertion into and manipulation within a patient's vascular system using techniques which are now well known in the medical community, so that the distal region of the catheter body may be brought to a desired location within the vascular system.

The overall dimensions of the catheter will depend on use, with the length varying widely, typically being between about 40 cm and 150 cm, usually being from about 40 cm to about 120 cm for peripheral catheters and being from about 110 cm to about 150 cm for coronary catheters. The diameter of the catheter body may also vary widely, with the diameter of the distal region typically being from about 2 F (French) to about 3 F, and the diameter of the proximal region typically being from about 3 F to about 6 F. Preferably the catheter has a substantially uniform diameter of from about 2 F to about 6 F, more preferably from about 3 F to about 6 F. Longitudinally extending lumens within the catheter will typically have a diameter of from about 0.1 mm to about 1.0 mm, preferably from about 0.2 mm to about 0.8 mm.

The recanalization or revasculariztion catheter may be comprised of a wide variety of biologically compatible materials, typically being made from natural or synthetic polymers such as silicone rubber, natural rubber, polyvinylchloride, polyurethanes, polyesters, polyethylene, polypropylene, polytetrafluoroethylene (PTFE), and the like. Frequently, the catheter may be formed as a composite having a reinforcement material incorporated within the elastomeric body to enhance strength, flexibility, and toughness. The flexible tubular members of the catheter body will normally be formed by extrusion, with two or more integral lumens being provided. The catheter diameter can then be modified by heat expansion and shrinkage using conventional techniques. Particular methods or techniques for forming the vascular catheters of the present invention are well described in the patent and medical literature.

A catheter body or shaft useful according to the invention may, for example, be formed from a single tubular member that extends the entire distance from the proximal end to the distal end, or it may be formed from two or more tubular members that are joined together, either in tandem or in parallel. On the catheter bodies formed from a single tubular member, the proximal region may be expanded relative to the distal region and appropriate lumens will be formed in the interiors of the two regions. Alternatively, the distal region in the catheter body may be formed from a single tubular member having two or more lumens while the proximal region is formed from a second tubular member having at least two axial lumens. The two regions may then be joined together so that the lumens and the distal tubular element are contiguous with both the parallel axial lumens and the proximal region. Further alternatively, the catheter body may include a single tubular member having two or more substantially longitudinal lumens which extend the entire length from the distal end to the proximal end. The proximal section is formed by securing a second tubular member to the side of the first tubular member and penetrating the first tubular member so that the respective lumens are made contiguous. The distal region of the catheter is that portion which remains forward of the point where the two tubes are joined.

The tapered distal tip of the catheter preferably comprises a rigid or semi-rigid metal or polymeric surface that is integral with or bonded, glued, or otherwise affixed to the distal end of the catheter. Guidewires useful with the system described herein would be known to those skilled in the art. However, it is understood that an operator would tend to select stiffer guidewires that would facilitate creating openings in or crossing an occlusion.

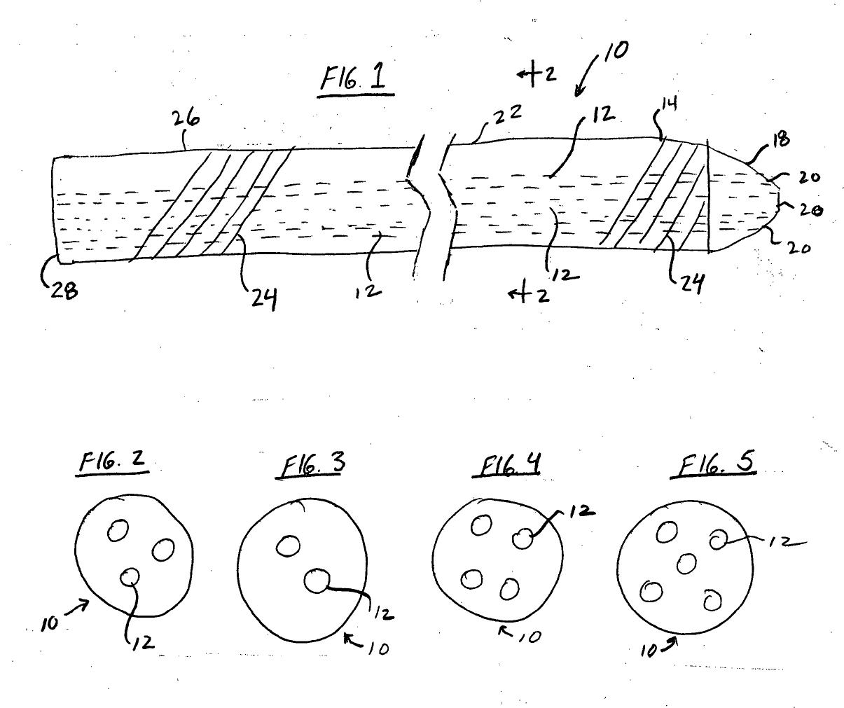

The invention can perhaps be understood by making reference to the drawings. In FIG. 1 a catheter 10 has two or more substantially parallel, longitudinally extending lumens 12, shown in dotted lines. The distal section 14 of catheter 10 terminates in a tapered distal tip 18, which has an opening 20 for each lumen 12.

In a preferred embodiment of the invention the outer surface 22 of catheter 10 has substantially parallel grooves or threads 24 that are at angle to the longitudinal axis of catheter 10. That angle could be from about 15° to 75°, preferably from about 30° to 60°, from the longitudinal axis. Grooves 24 are optionally positioned proximal to distal tip 18 but may also be located in the proximal section 26 of catheter 20.

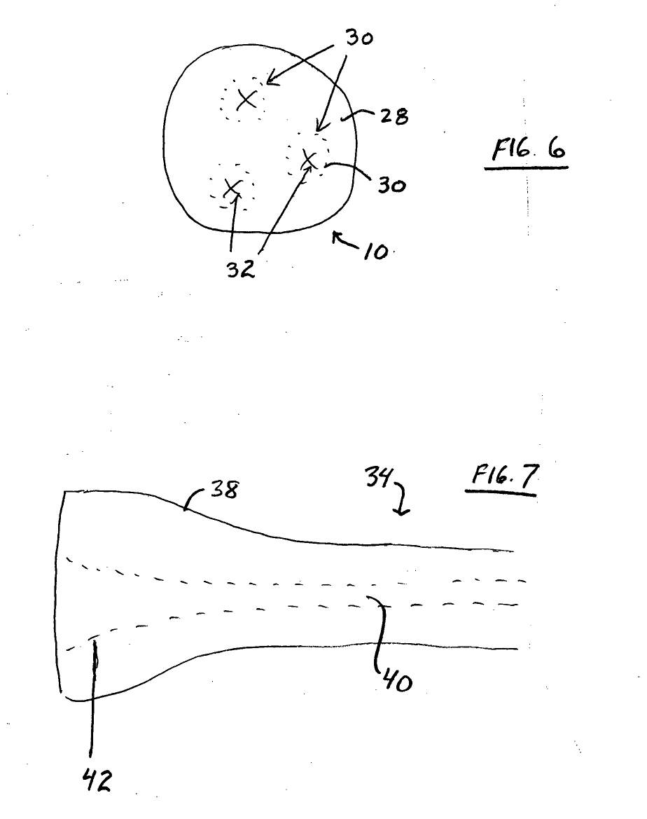

The embodiment of the invention shown in FIG. 1 has three lumens 12, which is reflected in the cross-sectional view of FIG. 2. The cross-sectional views of FIGS. 3 to 5 represent other embodiments with 2, 4, or 5 lumens, respectfully. These different permutations offer flexibility for different complexities of the chronic total occlusions, for example, choice of the large number of lumens for longer, more chronic occlusions, or chronic total occlusion with previously unsuccessful attempts.

FIG. 6 represents a view of the proximal end 28 of catheter 10. Each channel or lumen 12 has a one-way valve 30 (dotted lines) to prevent back bleeding, with slits 32 for insertion of a guidewire or a balloon catheter.

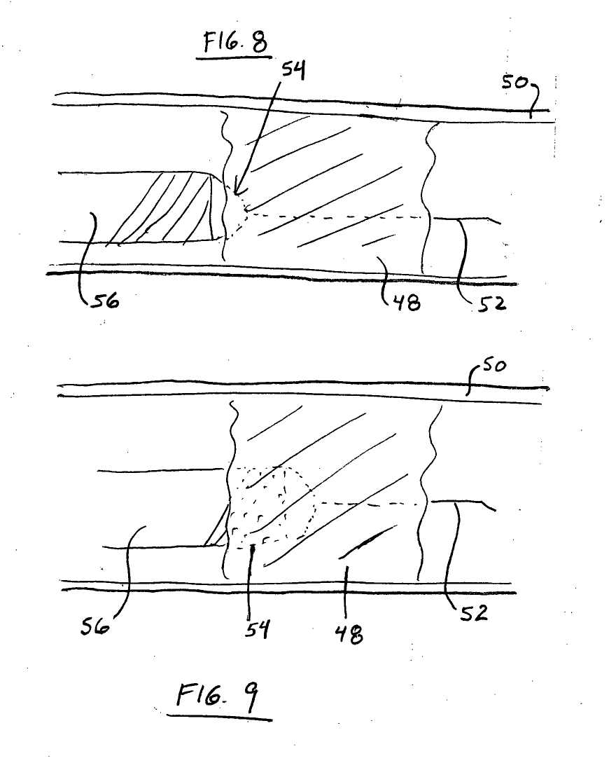

In FIG. 7 a catheter 34 has a flared proximal end 38. For illustrative purposes a distensible lumen 40 (dotted lines), has an expanded proximal position 42.

FIGS. 8 and 9 represent a partial cross-sectional view of the advancement of the device of the invention in a chronic total occlusion 48 of an artery 50 following successful crossing of occlusion 48 by a guidewire 52. In FIG. 8 the distal tip 54 of a catheter 56 is wedged in fibrocalcific occlusion 48 without completely crossing the occlusion. As shown in FIG. 9, catheter 56 has been torqued manually with forward pushing to propagate distal tip 54 of catheter 56 further into occlusion 48.

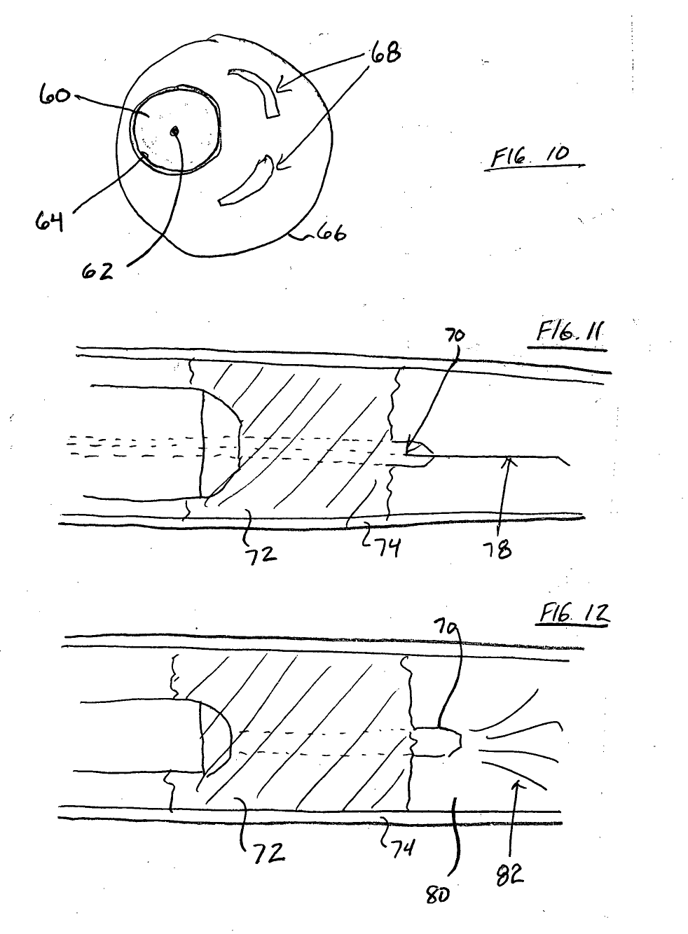

FIGS. 10 to 12 represent the insertion of a low-profile balloon catheter or other catheter over a guidewire in one of the lumens. FIG. 10 depicts a variation of the cross-section of FIG. 2 where a catheter or dilatation balloon catheter 60 has been advanced over guidewire 62 within a lumen 64 of a catheter 66. Lumen 64 has stretched or distended to accommodate catheter or dilatation balloon catheter 60 whereas lumens 68 have collapsed or been compressed to facilitate the distensibility of lumen 64 with catheter or dilatation balloon catheter 60. In the partially cross-sectional view of FIG. 11 a catheter or dilatation balloon catheter 70 has been advanced over a guidewire 78 beyond an occlusion 72 in an artery 74. In an additional step represented by FIG. 12, opacification of the distal vessel 80 is achieved by injecting contrast fluid 82 through the lumen remaining in catheter or dilatation balloon catheter 70 after temporary withdrawal of guidewire 78 and confirmation of the intravascular location. The “other” catheter could be a catheter with ultrasound, laser, RF, or abrasive capability capable of recanalization or revascularization.

The preceding specific embodiments are illustrative of the practice of the invention. It is to be understood, however, that other expedients known to those skilled in the art or disclosed herein, may be employed without departing from the spirit of the invention or the scope of the appended claims.

1. A catheter comprising a longitudinally extending cylindrical member and having a distal end, a proximal end, and at least two substantially parallel lumens extending therethrough, wherein the catheter is useful for recanalization of a total occlusion in a patient's vascular system.

2. The catheter of claim 1, wherein the distal end of the catheter has a tapered or bullet shape.

3. The catheter of claim 2, wherein the distal end of the catheter comprises a rigid or semi-rigid surface.

4. The catheter of claim 3, wherein the surface comprises a physiologically acceptable polymeric or metallic material.

5. The catheter of claim 1, wherein the catheter has an exterior surface with circumferential grooves.

6. The catheter of claim 5, wherein grooves are adjacent the distal end.

7. The catheter of claim 5, wherein grooves are adjacent the proximal end.

8. The catheter of claim 1, wherein the catheter has a proximal exterior surface with roughness, knobs, nubs, or other protrusions to facilitate torquing of the catheter.

9. The catheter of claim 1, wherein at least one of the lumens can be stretched or distended to accommodate a low-profile balloon catheter or other catheter.

10. The catheter of claim 1 which is adapted to enable a subsequent revascularization process requiring transversal of the occlusion by a guidewire.

11. A system for recanalization of a total occlusion in a patient's vascular system which comprises at least one guidewire and a catheter of claim 1.

12. The system of claim 11 which comprises at least two guidewires.

13. The system of claim 1 1, wherein at least one guidewire is stiffer than at least one other guidewire.

14. The system of claim 12, wherein there are two or three guidewires.

15. The system of claim 12, wherein there are three or four guidewires.

16. The system of claim 11, wherein the guidewires address bifurcation or trifurcation areas to facilitate “buddy” guidewire insertion.

17. The system of claim 11, which also comprises a low-profile balloon dilatation catheter or other catheter.

18. The system of claim 17, wherein the other catheter comprises ultrasound, laser, RF, or abrasive capability capable of recanalization and/or revascularization.

19. A method of recanalization of a total occlusion, which comprises:
advancing a guidewire percutaneously through a patient's vasculature to a point adjacent a total occlusion,
advancing a catheter of claim 1 over the guidewire to the point adjacent the occlusion, and
advancing the guidewire distally to cause the guidewire to cross the occlusion.

20. The method of claim 19 wherein the catheter is torqued.

21. The method of claim 19, wherein the total occlusion comprises atheromatic material.

22. The method of claim 19, wherein in an additional step a low-profile balloon dilatation catheter or other catheter is advanced over the guidewire through a lumen in the catheter and across the occlusion.

23. The method of claim 22, wherein the other catheter comprises ultrasound, laser, RF, or abrasive capability capable or recanalization and/or revascularization.

24. A method of recanalization of a total occlusion, which comprises:
advancing a first guidewire percutaneously through a patient's vasculature to a point adjacent a total occlusion,
advancing a catheter of claim 1 over the first guidewire to a point adjacent the occlusion, and
advancing a second guidewire through a lumen in the catheter and across the occlusion.

25. A method of recanalization of a total occlusion, which comprises:
advancing a first guidewire percutaneously through a patient's vasculature to a point adjacent a total occlusion,
advancing a catheter of claim 1 over the first guidewire to a point adjacent the occlusion,
advancing a second guidewire through a lumen in the catheter to a point adjacent the occlusion, and
advancing a third guidewire through a lumen in the catheter and across the occlusion.
